SEQUENCE LISTING (13 sequences)

SEQ ID NO: 1 - DNA, 51 bp, Artificial Sequence
  shRNA specific for mouse beta-2-microglobulin
gcctcacattgaaatccaaatttcaagagatatagaaagaccagtccttgc

SEQ ID NO: 2 - DNA, 51 bp, Artificial Sequence
  shRNA specific for mouse alpha-chain of H2E
gcgttccaacaacactccagattcaagagatctggagtgttgttggaacgc

SEQ ID NO: 3 - DNA, 47 bp, Artificial Sequence
  shRNA specific for human beta-2-microglobulin
gaatggagagagaattgaattcaagagattcaattctctctccattc

SEQ ID NO: 4 - DNA, 51 bp, Artificial Sequence
  shRNA specific for human CIITA
aagagaccagggaggcttatgttcaagagacataagcctccctggtctctt

SEQ ID NO: 5 - DNA, 220 bp, Artificial Sequence
  H1 promoter for transcription of shRNA
aattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccac

SEQ ID NO: 6 - DNA, 245 bp, Artificial Sequence
  EFS
tggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcg

SEQ ID NO: 7 - DNA, 1080 bp, Artificial Sequence
  EF1 alpha
catttcaggtgtcgtgaggaatttcgacatttaaatttaattaatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgaggtttaaactacgggatcttcgaaggcctaagcttacgcgcgcgtcctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgactagctagtagctagctagctagtcgagctcaagcttcggggactagtcatatgataatcaacctctggatta

SEQ ID NO: 8 - DNA, 11085 bp, Artificial Sequence
  empty vector backbone
ttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcgatgtcgacgataagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaatttcgacatttaaatttaattaatctcgacggtatcggttaacttttaaaagaaaaggggggattggggggtacagtgcaggggaaagaatagtagacataatagcaacagacatacaaactaaagaattacaaaaacaaattacaaaaattcaaaattttccgatcacgagactagcctcgaggtttaaactacgggatcttcgaaggcctaagcttacgcgcgcgtcctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgatagctagtagctagctagctagtcgagctcaacttcggggactagtcatatgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggtacgtatggccaggtacctttaagaccaatgacttacaaggcagctgtagatcttagccactttttaaaagaaaaggggggactggaagggctaattcactcccaacgaagacaagatgggatcaattcaccatgctagtggatccccctcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcggggctgcaggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacgcgtctccaagcttatcgataccgtcgcatgggaataacttcgtatagcatacattatacgaagttatgctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtag

SEQ ID NO: 9 - RNA, 51 nt, Artificial Sequence
  mouse shCIITA
gctgacctcccgtgtaaatgattcaagagatcatttacacgggaggtcagc

SEQ ID NO: 10 - DNA, 20 bp, Artificial Sequence
  shRNA for silencing HLA-DRA, HLA-DPA1 and HLA-DQA
gactgcagggtggagcactg

SEQ ID NO: 11 - DNA, 21 bp, Artificial Sequence
  shRNA for silencing HLA-DRA, HLA-DPA1 and HLA-DQA
tatgactgcaaggtggagcac

SEQ ID NO: 12 - DNA, 21 bp, Artificial Sequence
  shCIITA
aagagaccagggaggcttatg

SEQ ID NO: 13 - DNA, 264 bp, Artificial Sequence
  U6 promoter
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

(19) United States
(12) Patent Application Publication
(10) Pub. No.: US 2020/0197569 A1
(43) Pub. Date: Jun. 25, 2020

(54) GENETICALLY MANIPULATED CORNEA FOR TRANSPLANTATION

(71) Applicant: MEDIZINISCHE HOCHSCHULE HANNOVER, Hannover (DE)

(72) Inventors: Rainer Blasczyk, Isernhagen (DE); Constanca Ferreira De Figueiredo, Neustadt am Rübenberge (DE)

(21) Appl. No.: 16/715,983

(22) Filed: Dec. 16, 2019

(30) Foreign Application Priority Data
Dec. 21, 2018 (EP) 18215701.6

(51) Int. Cl. A61L 27/36; C12N 15/113

(52) U.S. Cl. CPC A61L 27/3687; A61L 27/3604; C12N 2310/14; C12N 2310/531; C12N 15/1138

(57) ABSTRACT

A cornea, which is ex vivo genetically manipulated in order not to elicit an immune response in an allogeneic recipient. The cornea is isolated, for use in transplantation and a method for production of a cornea, which is performed on an initial ex vivo, e.g. isolated cornea, for use in transplantation, e.g. for use in the treatment of cornea defects.

### PRIORITY CLAIM AND REFERENCE TO RELATED APPLICATION

The application claims priority of the European patent application EP18215701.6, filed on 21 Dec. 2018.

### FIELD

The present invention relates to an ex vivo genetically manipulated cornea for use in allogeneic transplantation, especially for use in transplantation to patients lacking the immune privilege of the eye, e.g. in patients who have developed adaptive or humoral immune response, e.g. who have previously received a cornea transplant, have neovascularization and/or an inflammation in the eye. Further, the invention relates to the ex vivo method of production of the genetically manipulated cornea for use in allogeneic transplantation, and to the use of nucleic acid constructs, e.g. viral vectors and viral particles, in the method of production.

### BACKGROUND

Figueiredo et al., Suppl. to Transplantation 1572 (2012) describes corneal transplants expressing shRNA directed against β2-microglobulin.

Figueiredo and Blasczyk, INTECH, p. 314 (2016) describe lentiviral genetic manipulation of allograft tissue to express shRNA directed against β2-microglobulin or against the α-chain of HLA-DR for decreasing immunogenicity of the tissue.

### SUMMARY

The invention provides a cornea, which is ex vivo genetically manipulated in order not to elicit an immune response in an allogeneic recipient. The cornea is isolated, for use in transplantation and a method for production of a cornea, which is performed on an initial ex vivo cornea, e.g. on an isolated cornea, for later use of the genetically manipulated cornea in transplantation, e.g. for use in the treatment of cornea defects. The genetically manipulated cornea is alive and due to the genetical manipulation is immunologically compatible with the later transplant recipient of the cornea. Accordingly, the cornea after being genetically manipulated is immunologically compatible to a transplant recipient to whom the original cornea was allogeneic. An advantage of the invention is to provide an alternative ex vivo genetically manipulated cornea for use in allogeneic transplantation and that preferably is less immunogenic, and an alternative ex vivo method of production of ex vivo genetically manipulating a cornea, wherein the method preferably produces a cornea which is less immunogenic.

### BRIEF DESCRIPTION OF THE DRAWINGS

FIG. 1 shows results of the analyses for corneas that were transduced with a lentiviral nucleic acid construct containing an expression cassette for one of shRNA specific for silencing β2-microglobulin (shβ2m), shRNA specific for silencing the alpha-chain of H2E (shH2Ea) or shRNA specific for silencing CIITA (shCIITA).

FIG. 2 shows histological analyses of the corneas in cross-sections in hematoxylin and eosin (HE) staining, in A of a freshly explanted mouse cornea, in B of a non-transduced cornea after two days of culture in organ culture medium at 37° C., and in C a cornea produced by the in vitro method of production for the genetic manipulation by transduction.

FIG. 3 shows the antibody levels in the sera as well as the tissue of the transplanted corneas of mice 9 weeks after transplantation.

FIGS. 4A and 4B respectively show the tissue analyses using Luminex technology and results of classifying infiltration by T-cells to scores 1 (little or no T-cell infiltration) to 4 (massive T-cell infiltration).

### DESCRIPTION OF THE INVENTION

The invention provides a cornea, which is ex vivo, e.g. isolated, for use in transplantation and a method for production of a cornea, which is performed on an initial ex vivo, e.g. isolated cornea, for use as a transplant, respectively for use in transplantation according to the claims. Accordingly, the cornea can be for use in the treatment of cornea defects. The cornea is genetically manipulated in order to only elicit a reduced immune response in an allogeneic recipient, preferably in order to not elicit an immune response in an allogeneic recipient. Specifically, the initial cornea is alive, e.g. an explanted cornea from an allogeneic donor, and kept under cell culture conditions, preferably in a serum-free cell-culture medium. This initial cornea is immunologically not compatible with the later recipient, e.g. the initial cornea is MHC-mismatched with the MHC of the later recipient. The initial cornea is isolated, e.g. maintained in organ culture medium. Optionally, the initial cornea is of non-human origin.

The cornea is genetically manipulated to contain a nucleic acid construct containing an expression cassette encoding inhibitory RNA sequences, e.g. siRNA, preferably short-hairpin RNA (shRNA), for use in human initial corneas is specific for the transcript of at least one of, preferably of all of the alpha or beta-chain of HLA-DR, DQ and DP molecules, respectively for murine initial corneas encoding inhibitory RNA sequences specific for the transcript of the alpha-chain of the H2E gene, and/or in each case encoding an inhibitory RNA (shRNA) specific for the transcript of the class II transactivator gene (CIITA), preferably further in combination with an expression cassette encoding an inhibitory RNA e.g. siRNA, preferably short-hairpin RNA (shRNA), specific for the transcript of the β2-microglobulin (β2m) gene. The cornea obtained by the process is for use as a transplant, e.g. as a replacement for a damaged cornea, in an immunologically non-compatible recipient, especially for use in a recipient whose MHC is not compatible to the MHC of the cornea initially used in the method for production.

The expression cassettes contain a promoter controlling the transcription of the encoded gene, preferably a constitutive promoter, e.g. the H1 promoter (SEQ ID NO: 5) or the U6 promoter (SEQ ID NO: 13) or the elongation factor short (EFS) promoter (SEQ ID NO: 6) or the EF1alpha promoter (SEQ ID NO: 7).

For introduction of the nucleic acid construct into the cornea, the nucleic acid construct can be contained in a viral vector, e.g. in a lentiviral vector, preferably packaged in lentiviral particles.

An ex vivo cornea containing an expression cassette encoding siRNA specific for the transcript of the alpha-chain or of the beta-chain of HLA class II genes, e.g. specific for the transcript of at least one of HLA-DR, DQ and DP molecules, preferably of all of the alpha-chain of HLA-DR, DQ and DP molecules and/or at least one of, preferably all of, the beta-chain of HLA-DR, DQ and DP molecules, and/or an expression cassette encoding siRNA specific for the transcript of the class II transactivator gene (CIITA), preferably also an expression cassette encoding siRNA specific for the transcript of β2m has been found to effect significantly less immunologic responses after transplantation, e.g. essentially no significant immunologic response after transplantation, even e.g. in recipients having neovascularizations in the eye, previous or present inflammation in the eye and/or previous contact with allogenic tissue, e.g. patients who have developed an immune response against allogenic HLA molecules, e.g. patients who had previously received an allogeneic transplant. Generally, the genetically manipulated cornea which is produced by the method of the invention can be transplanted as a cornea replacement into the eye of a patient.

The introduction of an expression cassette encoding siRNA specific for the transcript of the human alpha-chain or of the beta-chain of HLA class II gene for an initial human cornea, respectively encoding siRNA specific for the transcript of the murine alpha-chain of the H2E gene for an initial murine cornea, and/or, in each case, introduction of an expression cassette encoding siRNA specific for the transcript of the class II transactivator gene (CIITA) has been found to result in the reduction, respectively suppression of the translation of MHC II in the cornea, and introduction of an expression cassette encoding shRNA specific for the transcript of β2m has been found to result in the reduction, respectively suppression of the translation of MHC I in the cornea.

It was found that transplantation of a cornea expressing siRNA specific for the transcript of the alpha-chain of the H2E gene and/or expressing siRNA specific for the transcript of the class II transactivator gene (CIITA), preferably also expressing shRNA specific for the transcript of β2m, does not induce an immunogenic reaction by the recipient, e.g. no de novo formation of alloantibodies, and does not result in graft rejection in vivo. Preferably, the genetically manipulated cornea of the invention after transplantation does not elicit an immune response in the recipient.

An shRNA specific for the mouse β2m gene transcript (MM_shβ2m) can comprise or consist of SEQ ID NO: 1, corresponding to the nucleotide sequence of 51 nt (nucleotides) starting at nucleotide No. 209 of X01838 (European Nucleotide Archive), an shRNA specific for the mouse alpha-chain of the H2E gene transcript can comprise of consist of SEQ ID NO: 2, corresponding to the nucleotide sequence of 51 nt starting at nucleotide No. 324 of U13648.1 (European Nucleotide Archive).

An shRNA specific for the human β2m gene transcript (HS_shβ2m) can comprise or consist of SEQ ID NO: 3, corresponding to the nucleotide sequence of 47 nt (nucleotides) starting at nucleotide No. 244 of NM_004048.2 (NCBI databank), an shRNA specific for the human class II transactivator gene transcript (HS-shCIITA) can comprise of consist of SEQ ID NO: 4, corresponding to the nucleotide sequence of 51 nt starting at nucleotide No. 400 of NM_000246 at (NCBI databank).

The shRNA loop contained is TTCAAGAGA.

A preferred promoter for driving the transcription of the shRNA encoding sequences is the H1 promoter, e.g. comprising or consisting of SEQ ID NO: 5.

The elongation factor short (EFS) promoter preferably comprises or consists of SEQ ID NO: 6, the EF1-alpha promoter preferably comprises or consists of SEQ ID NO: 7.

An exemplary lentiviral vector backbone has SEQ ID NO: 8, which contains a shRNA cloning site at nt 5312, and 5629.

For simultaneously targeting and inhibiting HLA-DRA, HLA-DPA1 and HLA-DQA, shRNA of SEQ ID NO: 10 or of SEQ ID NO: 11 or a combination of these can be used, or a sequence having at least 80%, at least 85%, at least 90%, preferably at least 95% sequence identity.

For inhibiting CIITA, shRNA of SEQ ID NO: 12 (start nt 400, aa 90) can be used.

In the process for producing the genetically manipulated cornea, an explanted live cornea is washed, e.g. in serum-free organ culture medium. A preferred organ culture medium is Human Endothelial SFM (Gibco) or Culture Medium II (Biochrom) supplemented with 10 ng/ml FGF-2. The cornea is kept in serum-free organ culture medium and transfected with the nucleic acid construct containing the expression cassettes encoding siRNA specific for the transcript of the alpha-chain of the H2E gene and/or expressing siRNA specific for the transcript of the class II transactivator gene (CIITA), preferably also expressing siRNA specific for the transcript of β2m. Preferably, the nucleic acid construct is contained in a viral vector, e.g. in a lentiviral vector, packaged into lentiviral particles, and introduced into the medium. Following an incubation period, e.g. for 2h at 37° C. under cell culture conditions, the cornea is washed, e.g. in organ culture medium, and optionally incubated in fresh organ culture medium, e.g. for 48h at 37° C. Following this incubation, the cornea can be transplanted into a recipient.

### Example: Genetically Manipulating Mouse Cornea In Vitro for Use as a Transplant

Corneas from C3H/HeJ (MHC haplotype H-2^{k}) or from Balb/cJ (control, MHC matched) mice were explanted and transduced with lentiviral particles containing nucleic acid constructs encoding an expression cassette for shRNA specific for silencing mRNA for β2-microglobulin of SEQ ID NO: 1, and/or encoding an expression cassette for shRNA of SEQ ID NO: 9 (shRNA specific for the murine_shCIITA, NM_001302618.1 (start 992, aa304), GCTGACCTCCCGTGTAAATGA TTCAAGAGA TCATTTACACGGGAGGTCAGC) specific for silencing murine CIITA transcripts, and/or encoding shRNA of SEQ ID NO: 2 specific for silencing the murine alpha-chain of H2E. The expression cassettes were contained in a lentiviral vector according to SEQ ID NO: 8, which was packaged in lentiviral particles. For transduction, the corneas were incubated in organ culture medium (culture medium II, Biochrom) containing 8 μg/mL protamine sulfate for 2h at 37° C. Afterwards, the medium containing the viral was replaced with fresh organ culture medium and the corneas were incubated for 48h at 37° C. to produce the genetically manipulated corneas. Then, the genetically manipulated corneas (MHC haplotype H-2^{d}) were transplanted into new C3H/HeJ (MHC haplotype H-2^{k}) mice which were completely mismatched for MHC.

The organ culture medium could be replaced by cell culture medium.

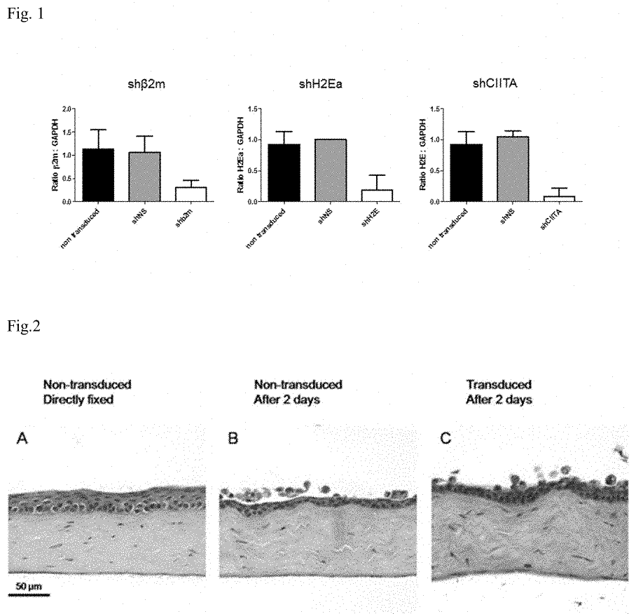

From the corneas that were genetically manipulated in vitro, resp. ex vivo, a section was taken and analysed by quantitative RT-PCR for transcripts of β2-microglobulin, of the alpha-chain of H2E, and of the CIITA. The results of the analyses for corneas that were transduced with a lentiviral nucleic acid construct containing an expression cassette for one of shRNA specific for silencing β2-microglobulin (shβ2m), shRNA specific for silencing the alpha-chain of H2E (shH2Ea) or shRNA specific for silencing CIITA (shCIITA) are depicted in FIG. 1, each normalized for the concentration of the mRNA for GAPDH. For comparison, non-transduced corneas and corneas transduced with an expression cassette encoding for the control shRNA shNS are shown. These results show that silencing of β2-microglobulin, of the alpha-chain of H2E, or of the CIITA was effective.

FIG. 2 shows histological analyses of the corneas in cross-sections in hematoxylin and eosin (HE) staining, in A of a freshly explanted mouse cornea, in B of a non-transduced cornea after two days of culture in organ culture medium at 37° C., and in C a cornea produced by the in vitro method of production for the genetic manipulation by transduction. These analyses show that the method of production essentially maintains the structure of the cornea.

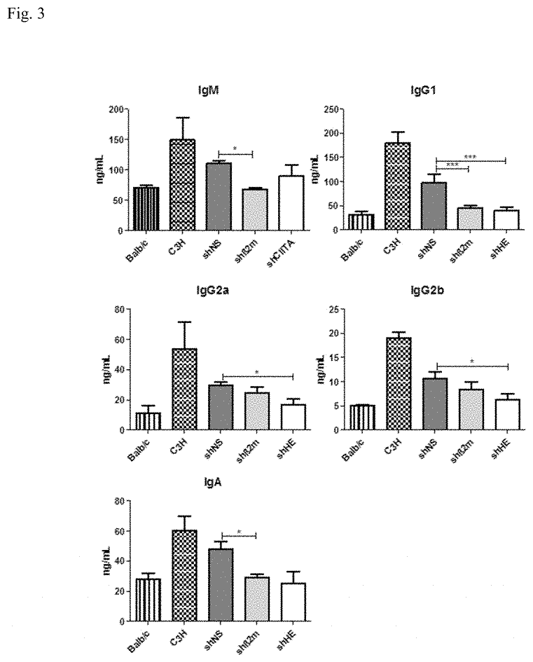

9 weeks after transplantation, mice were killed and the antibody levels in the sera as well as the tissue of the transplanted corneas were analysed. The results, which are depicted in FIG. 3, show a significant decrease in the levels of IgA, IgG1 and IgM antibodies in animals transplanted with a MHC class I silenced corneas. Interestingly, significant decreases in the levels of IgG1, IgG2a and IgG2b were detectable in the sera of animals transplanted with a MHC class II silenced cornea. Also, lower levels of IgA and IgM were detectable in these animals in comparison to those receiving non-modified corneas or expressing a non-specific shRNA. These data shows that both MHC class I and class II corneas are significantly less immunogenic and trigger a much weaker antibody mediated alloimmune response in comparison to fully MHC expressing corneas.

Analysis for T-cell infiltration showed that significantly (p<0.05) lower rates of T-cells were observed in MHC-mismatched corneas expressing one of shRNA specific for silencing β2-microglobulin, shRNA specific for silencing CIITA or shRNA specific for silencing the alpha-chain of H2E.

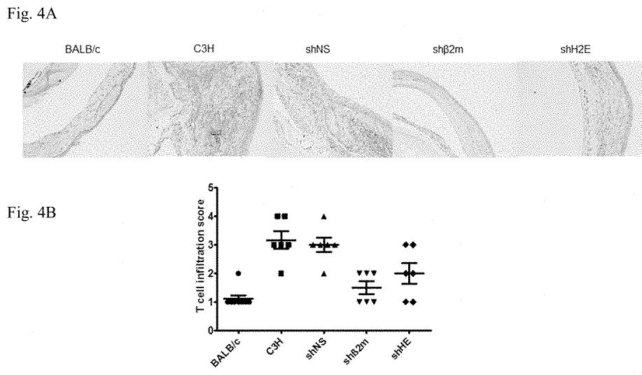

The results are shown in FIG. 4B, which were obtained from the tissue analyses of FIG. 4A using Luminex technology and classifying infiltration by T-cells to scores 1 (little or no T-cell infiltration) to 4 (massive T-cell infiltration).

FIG. 4B shows histological analyses of the corneas that were transplanted after 9 weeks in the recipient, in cross-sections with antibody staining for CD3 T-cells, and HE staining. For comparison, non-manipulated cornea from a Balb/c mouse that was transplanted into a Balb/c recipient mouse is shown, and a non-manipulated cornea transplanted and then retrieved from a C3H mouse, a C3H cornea expressing shNS, and the cornea that was genetically manipulated to express shRNA specific for silencing β2-microglobulin (shβ2m) and the cornea that was genetically manipulated to express shRNA specific for silencing the alpha-chain of H2E (shH2E). These results show that corneas silenced for β2-microglobulin showed a significantly decrease in immune cell infiltration and a better tissue structure. MHC class II silenced corneas according to the invention showed a tendency for decreased T-cell infiltration.

1. A cornea, ex vivo, for use in transplantation to a recipient that is immunologically non-compatible to an initial live isolated cornea, wherein the initial live cornea is genetically manipulated to contain an expression cassette expressing at least one inhibitory RNA specific for the alpha-chain and/or for the beta-chain of the MHC II gene and/or an expression cassette expressing an inhibitory RNA specific for the class II transactivator gene (CIITA).

2. The cornea for use in transplantation according to claim 1, wherein the cornea is genetically manipulated to additionally contain an expression cassette expressing an inhibitory RNA specific for the β2-microglobulin mRNA.

3. The cornea for use in transplantation according to claim 1, wherein the inhibitory RNA specific for the alpha-chain and/or for the beta-chain of the MHC II gene is specific for at least one of, preferably of all of the alpha-chain and/or beta-chain of HLA-DR, DQ and DP molecules.

4. The cornea for use in transplantation according to claim 1, wherein the initial cornea is of non-human origin or of human origin.

5. The cornea for use in transplantation in the treatment of a defective cornea according to claim 1.

6. A cornea, ex vivo, for use in transplantation to a recipient who is immunologically non-compatible to an initial live isolated cornea, wherein the initial live cornea in vitro is genetically manipulated to contain an expression cassette expressing at least one inhibitory RNA having a sequence of at least 80%, at least 85%, at least 90%, preferably at least 95% sequence identity to SEQ ID NO: 10 or to SEQ ID NO: 11, or a combination of these, for simultaneously targeting and inhibiting expression of HLA-DRA, HLA-DPA1 and HLA-DQA.

7. The cornea of claim 6, which is genetically manipulated to contain at least one expression cassette expressing an inhibitory RNA having a sequence of SEQ ID NO: 10 and at least one expression cassette expressing an inhibitory RNA having a sequence of SEQ ID NO: 11

8. A method for producing a cornea by genetically manipulating an initial isolated live cornea to contain an expression cassette expressing an inhibitory RNA specific for the alpha-chain and/or beta-chain of MHC II gene and/or an expression cassette expressing an inhibitory RNA specific for the class II transactivator gene (CIITA).

9. The method according to claim 8, wherein the inhibitory RNA specific for the alpha-chain and/or for the beta-chain of the MHC II gene is specific for at least one of, preferably of all of the alpha-chain and/or beta-chain of HLA-DR, DQ and DP molecules.

10. The method according to claim 8, wherein the cornea is genetically manipulated to additionally contain an expression cassette expressing an inhibitory RNA specific for the β2-microglobulin mRNA.

11. The method according to claim 8, wherein the initial cornea is maintained in serum-free organ culture medium, at least one nucleic acid construct containing the expression cassettes packaged in viral particles is added to the medium, the cornea is kept in the medium for an incubation period, and the medium is replaced by fresh serum-free medium for an incubation period sufficient for transcription of the expression cassettes.

12. The method according to claim 11, wherein the at least one nucleic acid construct which is added to the medium is a lentiviral vector which is packaged in lentiviral particles.

13. The method according to claim 8, wherein the genetically manipulated cornea produced is transplanted as a replacement cornea to the eye of a recipient.

14. A method for in vitro producing a cornea that is immunologically compatible to a recipient of the cornea as a transplant from a live cornea that in respect of the recipient is initially immunologically incompatible by genetically manipulating the initial isolated live cornea to contain an expression cassette expressing at least one inhibitory RNA having a sequence of at least 80%, at least 85%, at least 90%, preferably at least 95% sequence identity of SEQ ID NO: 10 or of SEQ ID NO: 11, or a combination of these, for simultaneously targeting and inhibiting expression of HLA-DRA, HLA-DPA1 and HLA-DQA.
